(19) United States
(12) Patent Application Publication
(10) Pub. No.: US 2024/0390329 A1
(43) Pub. Date: Nov. 28, 2024

(54) APPLICATION OF CHANNEL BLOCKER IN PREPARATION OF DRUGS FOR TREATING AND/OR PREVENTING LIVER FIBROSIS

(71) Applicant: Affiliated Hospital of Zunyi Medical University, Zunyi (CN)

(72) Inventors: Rui Xie, Zunyi (CN); Jingyu Xu, Zunyi (CN); Guowei Gong, Zunyi (CN); Qian Du, Zunyi (CN); Yanxia Hu, Zunyi (CN); Jianhong Ding, Zunyi (CN); Xianli Wu, Zunyi (CN); Xiaoxu Yang, Zunyi (CN); Jun Lou, Zunyi (CN); Qi Liu, Zunyi (CN); Zhuo Li, Zunyi (CN)

(21) Appl. No.: 18/462,388

(22) Filed: Sep. 6, 2023

(30) Foreign Application Priority Data
May 22, 2023 (CN) 2023105834920

(51) Int. Cl. A61K 31/4166; A61P 1/16

(52) U.S. Cl. CPC A61K 31/4166; A61P 1/16

(57) ABSTRACT

An application of a channel blocker in preparation of drugs for treating and/or preventing liver fibrosis is provided, belonging to the field of biomedical technology. A new use of phenytoin sodium is provided. It is demonstrated that at the cell level, the phenytoin sodium can improve liver fibrosis by regulating autophagy and thus affecting the activation of hepatic stellate cells. In this situation, the efficacy of the phenytoin sodium on liver fibrosis is verified at the animal level by establishing a disease model, and the phenytoin sodium can be used to prepare drugs for treating and/or preventing liver fibrosis.

### TECHNICAL FIELD

The disclosure relates to the field of medical technologies, and more particularly to an application/use of a channel blocker in preparation of drugs for treating and/or preventing liver fibrosis.

### BACKGROUND

Chronic liver disease is a worldwide problem that seriously affects people's quality of life. The morbidity and mortality of cirrhosis and primary hepatocellular carcinoma are very high, while liver fibrosis is the pathological basis of these chronic liver diseases and the pathogenesis of cirrhosis and hepatocellular carcinoma. A large number of studies have confirmed that liver fibrosis may be caused by a variety of reasons, including alcohol, viruses, drugs, toxins, immune and metabolic disorders, and the continuous stimulation of these substances may lead to serious liver damage. The pathogenesis of liver fibrosis is also related to many factors, and the activation of hepatic stellate cells (HSCs) is one of the key factors in its pathogenesis. Affected by many factors, persistent inflammatory necrosis occurs in hepatocytes, HSCs are activated and then proliferate in large quantities, and a large number of extracellular matrix (ECM) are released, which promotes the proliferation of fibrous connective tissue and eventually forms fibrous scars, thereby damaging the structure and function of the liver and eventually leading to liver fibrosis. Therefore, inhibition of the activation of HSCs is of great significance in the treatment of liver fibrosis.

At present, the treatment of liver fibrosis is still in its infancy. Although some effective drugs have been found, most of them are etiological treatment, which has the disadvantages of a long course of treatment, poor therapeutic effect and many adverse reactions, and its treatment has plagued the global medical community. In this situation, one of main tasks at present is to find the factors leading to liver fibrosis early, and intervene and inhibit its further development to chronic liver disease in time. Therefore, it is of great social and medical significance to study the pathogenesis of liver fibrosis, find new therapeutic targets, and develop safe, reliable and affordable drugs for the effective treatment of liver fibrosis.

### SUMMARY

A purpose of the disclosure is to provide an application of a channel blocker in preparation of drugs for treating and/or preventing liver fibrosis, to solve the problems existing in the related art.

To achieve the above purpose, the disclosure provides the following solutions.

The disclosure provides an application method of a channel blocker, includes: preparing a drug for treating and/or preventing liver fibrosis by using the channel blocker.

In an embodiment, the channel blocker is a sodium channel blocker.

In an embodiment, the sodium channel blocker is phenytoin sodium.

In an embodiment, the liver fibrosis includes carbon tetrachloride (CCl₄) induced liver fibrosis.

In an embodiment, the drug also includes pharmaceutically acceptable excipients.

In an embodiment, a dosage form of the drug is any medically recognized dosage form.

In an embodiment, the dosage form of the drug includes powder, an injection, a capsule, a tablet, and an oral liquid.

The disclosure also provides an application of phenytoin sodium in preparation of drugs to inhibit autophagy in liver tissue.

The disclosure also provides an application of phenytoin sodium in preparation of drugs to inhibit proliferation of hepatic stellate cells (HSCs).

The disclosure also provides an application of phenytoin sodium in preparation of drugs to downregulate alpha-smooth muscle actin (α-SMA) in hepatocytes.

The disclosure also provides an application of phenytoin sodium in preparation of drugs to downregulate microtubule-associated protein 1A/1B-light chain 3 (LC3) in hepatocytes.

The disclosure also provides an application of phenytoin sodium in preparation of drugs to upregulate protein 62 (p62, also referred to as sequestosome 1) in hepatocytes.

The disclosure discloses the following technical effects.

In the disclosure, it is demonstrated that at the cell level, the phenytoin sodium can improve liver fibrosis by regulating autophagy and thus affecting the activation of HSCs. In this situation, the efficacy of the phenytoin sodium on liver fibrosis is verified at the animal level by establishing a disease model, and the phenytoin sodium can be used to prepare drugs for treating and/or preventing liver fibrosis.

### BRIEF DESCRIPTION OF DRAWINGS

FIGS. 1A-1C illustrate effects of liver function of a mouse liver fibrosis model and mice treated by phenytoin (PHT) sodium. Specifically, FIG. 1A illustrates general morphology of liver in each group of mice; FIG. 1B illustrates a statistical diagram showing alanine aminotransferase (ALT) levels of mice in each experimental group; and FIG. 1C illustrates a statistical diagram showing aspartate aminotransferase (AST) levels of mice in each experimental group.

FIG. 2A illustrates liver fibrosis in mice of each group observed by hematoxylin and cosin (HE) staining.

FIG. 2B illustrates liver fibrosis in mice of each group observed by Masson staining.

FIG. 2C illustrates liver fibrosis in mice of each group observed by Sirius red staining.

FIGS. 3A-3B illustrate expression of alpha-smooth muscle actin (α-SMA) in each experimental group detected by immunohistochemistry.

FIGS. 4A-4F illustrate expression of a liver fibrosis index and an autophagy-related index in liver tissues of mice in each experimental group detected by Western blotting. Specifically, FIG. 4A and FIG. 4D illustrate expression of α-SMA in the liver tissue of mice in each experimental group. FIG. 4B and FIG. 4E illustrate expression of microtubule-associated protein 1A/1B-light chain 3 (LC3) in the liver tissue of mice in each experimental group. FIG. 4C and FIG. 4F illustrate expression of protein 62 (p62) in the liver tissue of mice in each experimental group.

FIG. 5 illustrates effects of PHT sodium on proliferation of hepatic stellate cells (HSCs) detected by cell counting kit-8 (CCK-8).

FIG. 6 illustrates effects of transforming growth factor beta 1 (TGF-β1) on proliferation of HSCs and effects of PHT sodium on TGF-β1 induced proliferation of HSCs detected by the CCK-8.

FIGS. 7A-7F illustrate expressions of α-SMA, LC3, and p62 of cells in different experimental groups detected by Western blotting. Specifically, FIG. 7A and FIG. 7D illustrate expression of α-SMA in HSCs of each experimental group. FIG. 7B and FIG. 7E illustrate expression of LC3 in HSCs of each experimental group. FIG. 7C and FIG. 7F illustrate expression of p62 in HSCs of each experimental group.

FIGS. 8A-8F illustrate expressions of α-SMA, autophagy-related proteins LC3, and p62 in HSCs after addition of rapamycin. Specifically, FIG. 8A and FIG. 8D illustrate expression of α-SMA in HSCs. FIG. 8B and FIG. 8E illustrate expression of LC3 in HSCs. FIG. 8C and FIG. 8F illustrate expression of p62 in HSCs.

### DETAILED DESCRIPTION OF EMBODIMENTS

Various exemplary embodiments of the disclosure will be described in detail, which should not be considered as a limitation of the disclosure, but should be understood as a more detailed description of certain aspects, features, and embodiments of the disclosure.

### Embodiment 1 Animal Experiment of Phenytoin (PHT) sodium

1. Construction and treatment of a mouse model of liver fibrosis:

1.1 Experimental procedures and methods:

(1) Experimental groups include a model group, a treatment group, a normal control group (also referred to as normal group), and a drug group, with a total of 60 mice for 15 healthy male C57 mice per group (purchased from Chongqing Tengxin Biotechnology Co., Ltd.).

(2) The mouse model for liver fibrosis is constructed when the weight of male C57 mice raised normally reaches 20 grams. The mice in the model group and the treatment group are intragastrically administered olive oil solution with 20% carbon tetrachloride (CCl₄), 5milliliters per kilogram (mL/kg), twice a week, for a total of 8 weeks. All mice are raised normally.

(3) Prevention and treatment of PHT sodium: after the second week of modeling, the mice in the treatment group and the drug group are intraperitoneally injected with PHT sodium (1 millimole per liter abbreviated as mM), 10 mL/kg, once a day, until the model group is successfully modeled. The mice in the normal control group and the model group are intraperitoneally injected with the same volume of physiological saline as PHT sodium.

(4) Sampling: after successful modeling, the mice in each group are dissected, and eyeballs are taken to measure serological indicators. The liver is taken to observe its appearance with the naked eye and photographed. 2 pieces of liver tissue are taken and fixed in formalin, and embedded in paraffin. Finally, paraffin sections are made for hematoxylin and cosin (HE) staining, picric-Sirius red staining. Masson's trichrome staining and immunohistochemistry, and the remaining tissues are frozen at −80 Celsius degree (° C.) for later use.

(5) HE staining of liver sections: the liver tissue of mice is fixed with formalin and embedded in paraffin. The paraffin sections are made by using adhesive slides to make paraffin sections, placed in an incubator at 70° C., baked for 1-2 hours, and subjected to conventional dewaxing treatment (soak the paraffin sections in xylene I solution for 10 minutes, xylene II solution for 10 minutes, anhydrous ethanol for 5 minutes, 95% ethanol for 5 minutes, 80% ethanol for 5 minutes, 75% ethanol for 5 minutes, 50% ethanol for 5 minutes, and finally rinse the paraffin sections three times with phosphate-buffered saline (PBS) buffer solution, each time lasting for 10 minutes). Then, the dewaxed sections are subjected to staining with hematoxylin for 5 minutes, differentiated with hydrochloric acid and alcohol for 10 seconds, then rinsed with running tap water for 10 minutes until the section become blue, then stained with 0.5% cosin solution for 3 minutes, and rinsed with running tap water for 5 minutes. Finally, the samples are dehydrated with 50% ethanol for 1 minute, 75% ethanol for 1 minute, 80% ethanol for 1 minute, 95% ethanol for 1 minute and anhydrous ethanol for 1 minute, sealed with neutral gum, observed under a microscope, and photographed and saved.

(6) Masson's trichrome staining of liver sections: the paraffin sections of the liver are subjected to dewaxing treatment, and the prepared Weigert's iron hematoxylin dye is dropped onto the tissue and stained for 5-10 minutes, followed by differentiation using acidic ethanol differentiation solution for 5-15 seconds and rinsing with water, then Masson blue staining solution (i.e., aniline blue) is used to become blue for 3-5 minutes, and distilled water is used to rinse for 1 minute. The rinsed sections are stained with the Ponceau fuchsin staining solution for 5-10 minutes, a weak acid working solution is prepared according to a ratio of distilled water to a weak acid solution of 2:1, and the stained sections are rinsed with the weak acid working solution for 1 minute. The sections after rinsing of the weak acid working solution are rinsed with a phosphomolybdic acid solution for 1-2 minutes, weak acid working solution for 1 minute, aniline blue staining solution for 1-2 minutes, weak acid working solution for 1 minute, 95% ethanol for rapid dehydration, anhydrous ethanol for 3 times, 5-10 seconds each time, xylene for clearing for 3 times, 1-2 minutes each time, sealed with neutral gum, and observed and photographed under the microscope.

(7) Picric-Sirius red staining of liver sections: in a clean environment, the paraffin sections of the liver are dewaxed and immersed in a hematoxylin solution for 2 minutes, then the sections are rinsed with tap water for 10 minutes, differentiated in 1% hydrochloric acid ethanol for 5 seconds, rinsed with tap water for 10 minutes, and immersed in picric-Sirius red staining solution for 20 minutes and then rinsed with tap water for 5 minutes. Finally, the processed sections are dehydrated, sealed, and finally observed and photographed under the microscope.

(8) Immunohistochemistry: the paraffin sections of the liver are dewaxed, immersed in a 3% H₂O₂ solution and incubated for 5-10 minutes, rinsed with distilled water and then rinsed with the PBS solution for 5 minutes, repeating twice. The antigen is repaired with sodium citrate and washed with the PBS solution for 5 minutes. The alpha-smooth muscle actin (α-SMA) antibodies are added dropwise and kept overnight at 4° C. After rewarming for 30 minutes, the antibodies are rinsed with the PBS solution for 5 minutes each time, repeating 3 times. The enzyme labeled monoclonal mouse/rabbit antibodies IgG are added dropwise, incubated at 37° C. for 30 minutes, and then rinsed with the PBS solution. The developer is used to develop color for 1 minute (3,3′-diaminoben-zidine abbreviated as DAB), then thoroughly rinsed with clean water, re-stained, dehydrated, cleared, and finally sealed, and finally photographed under the microscope and analyzed.

(9) Western blot experiment

1) Extraction of protein stock solution

Extraction of tissue protein: the liver tissue of mice in each experimental group is taken and placed into Eppendorf (EP) tubes and lysis buffer and protease inhibitor are added in proper proportion, mixed evenly, and the cells and nucleic acids are ultrasonically lysed to make the solution clear and transparent. The lysed products are centrifuged at a speed of 12,000 revolutions per minute (rpm) for 30 minutes at 4° C., and the supernatant is taken for protein quantification.

Extraction of cell protein: the cells that need to be extracted protein are removed from the incubator, rinsed twice with the prepared PBS solution, placed on a clean filter paper, and the remaining PBS is dried. Then, a certain amount of radio immunoprecipitation assay (RIPA) protein lysis solution is added according to the growth of the cells, and the protein is lysed for about 30 minutes. Then, the lysed protein stock solution is scraped with a scraper, placed in prepared EP tubes, and then placed in a high-speed centrifuge and centrifuged at 12,000 rpm at 4° C. for 30 minutes. After centrifugation is completed, the supernatant is taken for subsequent protein quantification.

2) Protein quantification

An EP tube is taken and added with 25 microliters (μL) standard protein bovine serum albumin (BSA) and 75 μL PBS to prepare 0.5 mg/mL BSA standard protein solution. A 96-well plate is taken and added with a certain amount of PBS and BSA to each well to set a standard curve. Then, the protein stock solution is diluted, 87 μL of PBS solution is added into the EP tube, and then 3 μL of centrifuged protein stock solution (diluted 30 times) is added. Then, the reagent A is mixed with the reagent B to prepare a bicinchoninic acid (BCA) working reagent at a concentration ratio of the reagent A to the reagent B of 50:1. 200 μL is added to each well in the 96-well plate, and then placed in a constant temperature box for 30minutes at 37° C. Finally, the concentration of the protein stock solution is detected with a microplate reader. After the concentration ratio is calculated, the RIPA protein lysis solution is added to the protein stock solution, the protein stock solution is adjusted to the same concentration, the same amount of 5 times the sample loading buffer is added to each EP tube, boiled in boiling water for 5 minutes, and put into the −20° C. refrigerator after cooling.

3) Experimental procedures of Western blotting

a. Electrophoresis gel is prepared.

b. Loading and running gel: the prepared electrophoresis gel is placed into the electrophoresis chamber, the electrophoresis solution is added, a sample loading comb is pulled out, the protein marker is taken out from the refrigerator, and 4 μL of the protein marker is added into the well, and the target proteins are added into the other wells. The lid is covered, the voltage is adjusted to 80 voltages (V) to start gel running, and when the band of the protein Marker is separated into multiple bands, the voltage is adjusted to 120 V until the position of the desired target protein is run out, and then the electrophoresis is terminated.

c. Membrane transfer: a polyvinylidene difluoride (PVDF) membrane, cut to the required size, and immersed in methanol solution for 5-10 minutes. The gel is taken from the electrophoresis tank, and the required gel is cut according to the molecular weight of the target protein and compared with the Marker band. The marked PVDF membrane is covered with the corresponding gel, placed the filter paper and sponge sequentially, then placed in the transfer tank, the transfer liquid is added, the voltage is adjusted to 90 V, and the transfer time is set according to the molecular weight of the target protein.

d. Blocking: after the electro transfer is completed, the PVDF membrane is removed, rinsed with Tris Buffered Saline with Tween® 20 (TBST) for 5 minutes, then the TBST is poured out, the prepared 5% protein blocking solution is added, and shaken in a shaker for 1.5 hours.

e. Incubation of primary antibodies: the blocked PVDF membrane is rinsed with TBST three times, each time for 10 minutes, and then the membrane is transferred to the prepared primary antibodies overnight.

f. Incubation of secondary antibodies: the PVDF membrane is taken out from the primary antibodies, rinsed with TBST three times, each time for 10 minutes, and then shaken in the secondary antibodies for 2 hours. The shaken PVDF membrane is taken out, and then rinsed with TBST three times, and each time for 10 minutes.

g. Color development: the developer is added dropwise onto the membrane and exposed.

h. Result analysis: the software Image J is used to statistically analyze the grayscale values of the exposure results.

1.2 Experimental results

(1) During the modeling process, the mental state of mice in the model group is gradually deteriorated, the activity is decreased, and the food intake and drinking water are decreased, indicating that the presence of CCl₄ can cause liver damage, thus affecting the healthy growth of the mice. Compared with the model group, the fur of the mice in the treatment group is still not smooth enough, but the activity of the mice is significantly increased, while the mice in the normal control group and the drug group have smooth fur and active activity, and the food intake and water intake are normal.

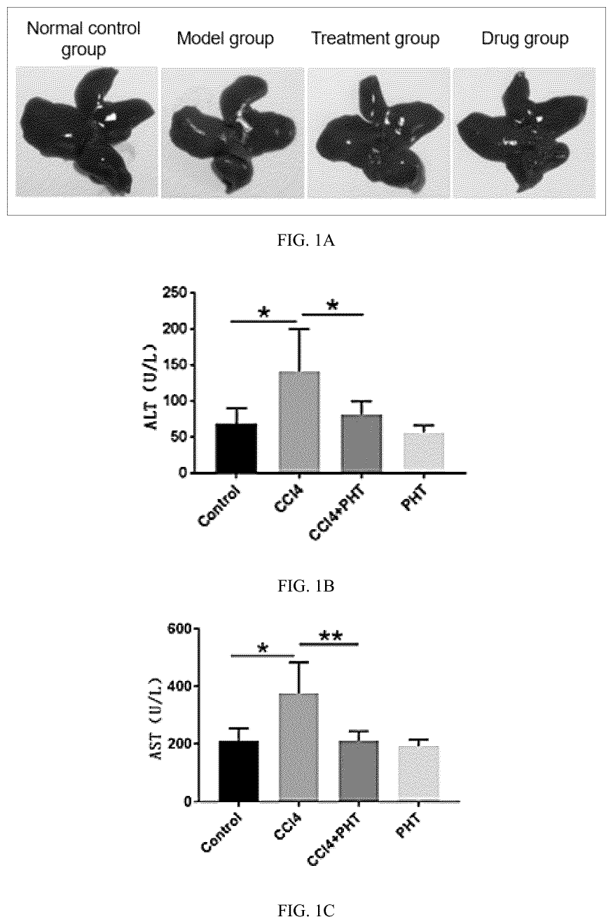

(2) After successful modeling, the mice are dissected, the appearance of the liver is observed with the naked eye and photographed. to record. As shown in FIG. 1A, it is evident that there are differences and changes in the appearance of the liver in each group of mice. There is no significant difference in the liver of mice between the normal control group and the drug group, and the liver has a soft texture and smooth surface. The liver of mice in the model group has a hard texture, low glossiness, rough surface, and the entire liver turns yellow. The liver texture of the treatment group mice is softer than that of the model group, but still harder than that in the normal group, and glossiness is low.

(3) Liver function is detected (alanine aminotransferase abbreviated as ALT, aspartate aminotransferase abbreviated as AST). The eyeball blood of mice in each group is taken and centrifuged to collect serum, and the serum is sent to the hospital laboratory to measure ALT and AST with instruments. As shown in FIG. 1B and FIG. 1C, the transaminase of the model group is higher than that of the normal control group, indicating that the liver fibrosis model is successfully constructed. The transaminase in the treatment group is lower than that in the model group, the difference is statistically significant (p<0.05), suggesting that PHT sodium could improve liver fibrosis. There is no significant difference in transaminase between the drug group and the normal control group, suggesting that the drug has no damage to the liver.

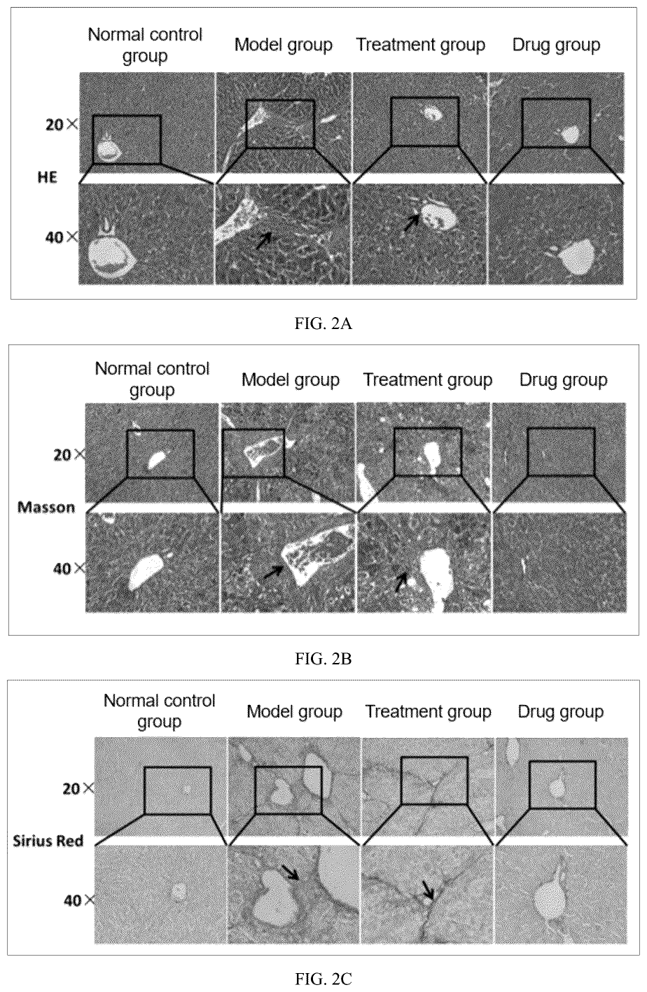

(4) Through the HE staining, Masson's trichrome staining, and picric-Sirius red staining of the liver sections, it can be found that the liver lobules of the mice in the normal control group and the drug group are intact and structurally normal, while in the model group of mice, a large amount of collagen deposition is observed around the portal area of the liver tissue, fibrous septa are formed between the portal area and the central vein, and the structure of liver lobules was disordered. Compared with the model group, the degree of liver fibrosis in the treatment group is reduced, as shown in FIGS. 2A-2C. It is indicated that PHT sodium can reduce the collagen content in the liver of mice and alleviate the degree of liver fibrosis.

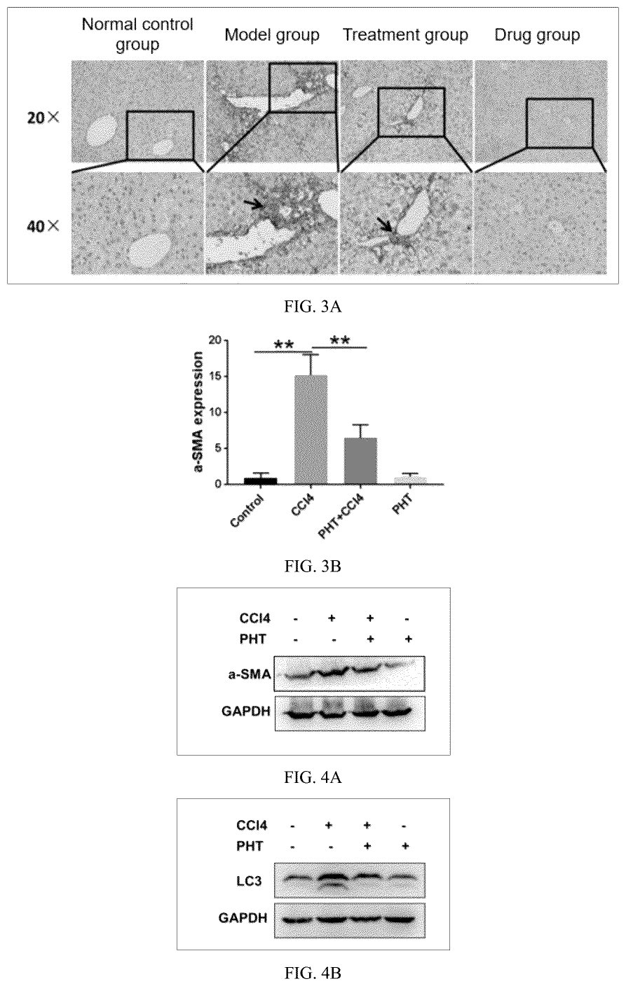

(5) The expression of α-SMA in liver sections of tissues of different experimental groups is detected by immunohistochemistry. As shown in FIG. 3, there is only a small amount of positive expression at the edges of hepatic portal vein and a small amount of central vein in the normal control group and the drug group, and there is a large amount of positive expression in the model group around portal area and inflammatory necrosis area in lobule and in the wall of hepatic sinus. The positive expression in the treatment group is decreased compared with the model group, and the results are statistically significant (p<0.05).

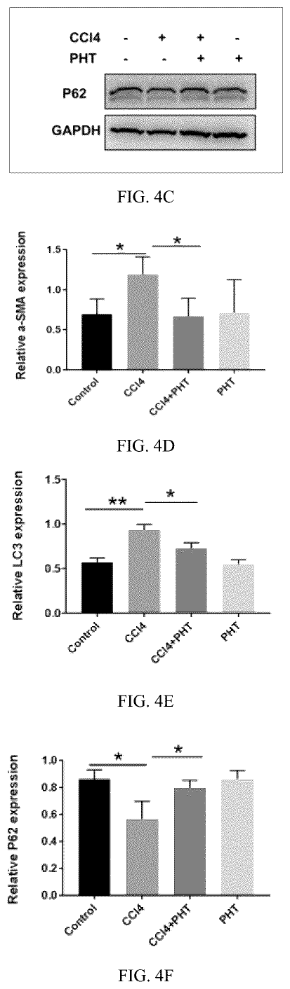

(6) The expressions of α-SMA, LC3, and p62 of tissues in different experimental groups are detected by using Western blotting. Compared with the normal control group, the expressions of liver fibrosis index α-SMA and autophagy-related index LC3 of liver tissue in the model group are increased, while the expression of p62 decreased. Compared with the model group, the expressions of liver fibrosis index α-SMA and autophagy-related index LC3of liver tissue in the treatment group are decreased, while the expression of p62 is increased, and the difference is statistically significant (p<0.05), as shown in FIGS. 4A-4F. The above results indicate that PHT sodium can improve liver fibrosis, which may be related to the autophagy pathway.

### Embodiment 2 Cell Experiment of PHT Sodium

1. Validation of the effect of PHT sodium on liver fibrosis at cell level

1.1 Experimental methods

(1) Cell culture

1) Cell recoveries are performed as follows. After irradiation with an ultraviolet lamp for 30 minutes on a super clean bench, white light and circulating air flow are turned on, a centrifuge tube is taken, and 3 mL of culture medium is added into the centrifuge tube. The frozen cells are quickly taken out of a refrigerator at −80° C. and put into a constant-temperature water bath at 37° C. to be thawed, and then the cells are sucked out from the super clean bench and put into the prepared centrifuge tube. After sealing, the cells are centrifuged in a centrifuge for 5 minutes (at a speed of 1000 rpm), then a culture flask is prepared, the name and date of the cells are marked, the supernatant is poured out after centrifugation, and a new culture medium is added to blow and mix. Finally, the mixed cells are added to the marked culture flask, and the flask is sprayed with 75% alcohol and placed in a constant temperature incubator for culture.

2) Cell passage is performed as follows. A Dulbecco's modified eagle medium (DMEM) complete culture medium, trypsin, and a sterile PBS are placed in a super clean bench, rewarm for 30 minutes, a white light and circulating airflow are turned on, an alcohol burner is lighted, the cells that need to be passaged are taken out from the incubator, the culture medium is poured out, sterile PBS is added for cleaning twice, then PBS is discarded, 1 mL of trypsin is added to the flask, and observed under a microscope. When the interval between cells becomes larger and the cells become round, the trypsin is immediately poured out, 3-5 mL culture medium is added into the flask to stop digestion, and then the cells are blown with a pipette. The blown cells are evenly packed into new culture flasks, and the name, passage times and passage duration of the cells are marked on the flasks. Finally, the flasks are sprayed with 75% alcohol and placed in a constant temperature incubator for continued culture.

3) Cryopreservation of cells is performed as follows. The required reagents are rewarmed. The cells that need to be frozen are taken out from the incubator, washed twice with sterile PBS, then PBS is discarded, and trypsin is added to digest the cells to a certain extent, 3-5 mL culture medium is added to the flask to stop digestion, and then the cells are blown with a pipette. Then, the blown cells are transferred to a new centrifuge tube, centrifuged, and the supernatant is poured out after centrifugation, 0.5-1 mL of frozen storage solution is added to the centrifuge tube and blown until well mixed. Finally, the mixed solution is added to a marked frozen storage tube, seal it, and stored in a −80° C. refrigerator.

(2) CCK-8 experiment

The cells in good condition are digested, and then the cells are counted. After counting the concentration ratio, the corresponding volume of cell suspension and culture medium are taken to make the cell concentration required for the experiment. The cells are fully mixed and added to a 96-well plate. According to the experimental requirements, different interventions are given to the cells. After 48 hours, 10% CCK8 solution (the ratio between culture medium and CCK8 is 10:1) is added to each well. After 1-4 hours of incubation in the incubator, the absorbance is detected by a microplate reader.

(3) Western-blot experiment is the same as the animal experiment in the embodiment 1.

1.2 Experimental results

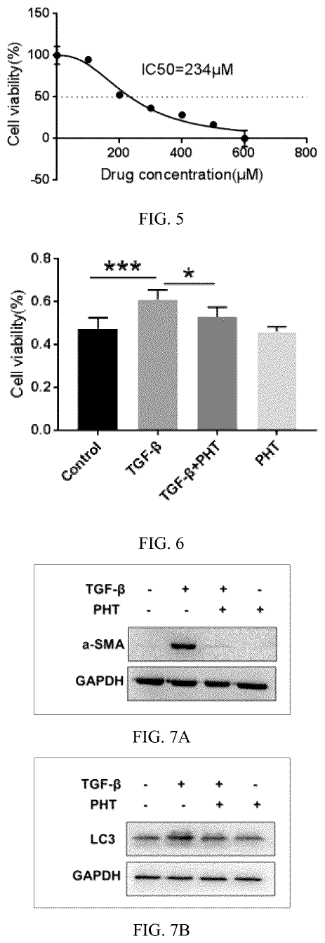

(1) CCK-8 method for detecting the effect of PHT sodium on the proliferation of HSCs and calculating the half-maximal inhibitory concentration (IC50) value: the effect of PHT sodium on the proliferation of HSCs is detected by the CCK8 method, and the proliferation inhibition rate of different concentrations of PHT sodium on HCSs is calculated. Finally, the IC50 value of PHT sodium on HSC is 234 micromoles per liter (μM), as shown in FIG. 5.

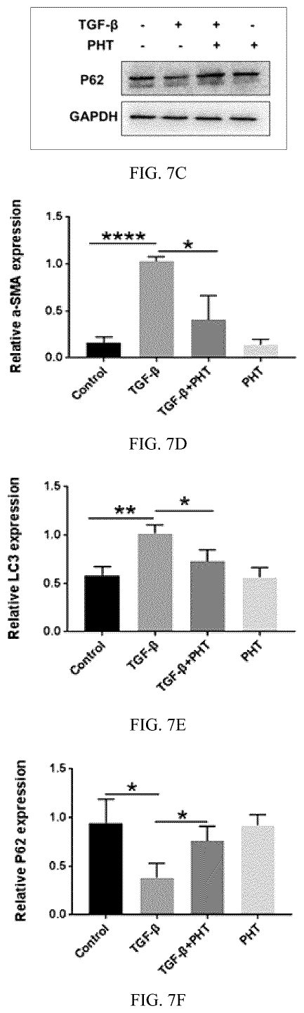

(2) CCK-8 method for detecting the effect of PHT sodium on the proliferation of HSCs induced by TGF-β1: the concentration of the drug is determined according to the IC50 value, and the concentration of PHT sodium is selected as 200 μM for cell-level related experiments. The effect of TGF-β1 on the proliferation of HSCs is detected by the CCK8 method. The results show that compared with the normal control group, TGF-β1 stimulation promotes the proliferation of HSCs, while after adding PHT sodium, the proliferation of HSCs promoted by TGF-β1 is inhibited. The results are statistically significant (p<0.05), as shown in FIG. 6.

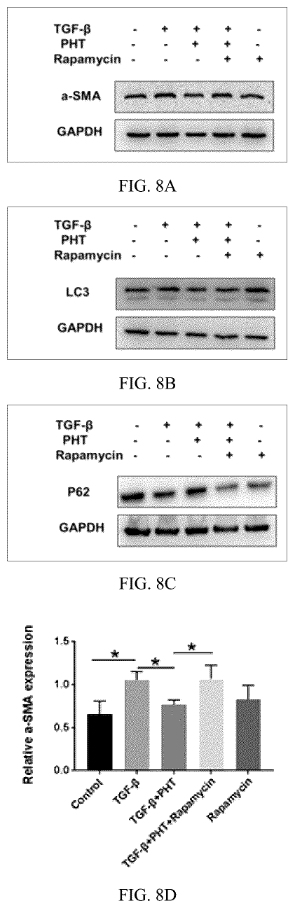

(3) The expressions of α-SMA, LC3 and P62 in different experimental groups are detected by Western blotting. As shown in FIGS. 7A-7F, the expression of α-SMA in the co-stimulation group of TGF-β1 and PHT sodium (i.e., TGF-β1+PHT) is significantly lower than that in the TGF-β1 stimulation group alone, and the difference is statistically significant (p<0.05). In addition, the results of the Western-blot experiment show that compared with the TGF-β1 stimulation group alone, the expression of autophagy-related protein LC3 is significantly decreased under the co-stimulation of PHT sodium and TGF-1, while the expression of autophagy substrate protein p62 is significantly increased, and the difference is statistically significant (p<0.05). These results indicate that TGF-β1 can promote the occurrence of autophagy during HSC activation, and the co-culture of PHT sodium and TGF-β1may hinder the occurrence of autophagy during HSC activation and inhibit the activation of HSCs, thus affecting the occurrence and development of liver fibrosis.

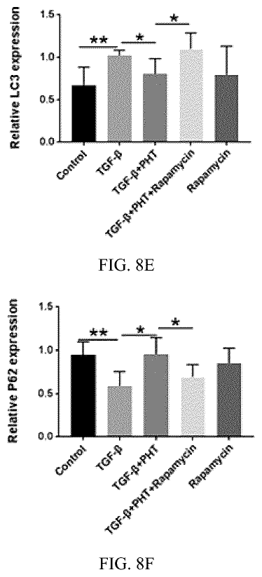

(4) It is confirmed that PHT sodium reverses the activation of HSCs by inhibiting autophagy by Western blotting. As shown in FIGS. 8A-8F, the expressions of α-SMA and autophagy-related protein LC3 are decreased while the expression of P62 is increased after HSCs are co-cultured with PHT sodium and TGF-β1. After the addition of autophagy agonist rapamycin, compared with PHT sodium and TGF-β1 group, the expressions of α-SMA and autophagy-related protein LC3 are increased in rapamycin, PHT sodium and TGF-β1 group (i.e., TGF-1+PHT+Rapamycin), while the expression of autophagy substrate protein P62 is decreased, and the difference is statistically significant (p<0.05). The results show that the results are restored after the addition of autophagy agonists, indicating that PHT sodium affects the activation of HSCs by regulating the autophagy pathway, thereby improving liver fibrosis.

The above-described embodiments are only a description of the illustrated method of the disclosure and do not limit the scope of the disclosure. Without departing from the design spirit of the disclosure, various modifications and improvements made by those skilled in the art to the technical solution of the disclosure shall fall within the scope of the disclosure.

What is claimed is:

1. A application method of a channel blocker, comprising:
preparing a drug for at least one of treatment and prevention of liver fibrosis by using the channel blocker.

2. The application method according to claim 1, wherein the channel blocker is a sodium channel blocker.

3. The application method according to claim 2, wherein the sodium channel blocker is phenytoin (PHT) sodium.

4. The application method according to claim 1, wherein the liver fibrosis comprises carbon tetrachloride (CCl₄) induced liver fibrosis.

5. The application method according to claim 1, wherein the drug comprises pharmaceutically acceptable excipients.

6. The application method according to claim 5, wherein a dosage form of the drug is any medically recognized dosage form.

7. The application method according to claim 6, wherein the dosage form of the drug comprises powder, an injection, a capsule, a tablet, and an oral liquid.

8. The application method according to claim 3, comprising:
using the phenytoin sodium to prepare a drug to inhibit autophagy in liver tissue.

9. The application method according to claim 3, comprising:
using the phenytoin sodium to prepare a drug to inhibit proliferation of hepatic stellate cells.

10. The application method according to claim 3, comprising:
using the phenytoin sodium to prepare a drug to downregulate alpha-smooth muscle actin (α-SMA) in hepatocytes.
